(19) United States
(12) Patent Application Publication
(10) Pub. No.: US 2014/0042239 A1
(43) Pub. Date: Feb. 13, 2014

(54) FRAGRANCE CONTAINER

(71) Applicant: SEN-MAO YEH, TAOYUAN (TW)

(72) Inventor: SEN-MAO YEH, TAOYUAN (TW)

(21) Appl. No.: 13/790,185

(22) Filed: Mar. 8, 2013

(30) Foreign Application Priority Data
Aug. 10, 2012 (TW) 101215475

(51) Int. Cl. A61L 9/12

(52) U.S. Cl. CPC A61L 9/125
USPC 239/34

(57) ABSTRACT

Disclosed is a fragrance container including a container body and a cover. The container body includes a plurality of chambers, each for containing fragrance. The cover is movably installed to the container body and provided for opening and closing each chamber of the container body, and the cover has a guide passage communicated to the outside and movable to a position corresponding to each chamber separately. Since the container body can contain fragrances of different smells in each chamber and the guide passage of the cover can be moved to a position corresponding to each chamber of the container body, the fragrances in different chambers can be dispersed to the outside to achieve the effects of providing users a choice of fragrances and a diversified application at appropriate time conveniently.

### BACKGROUND OF THE INVENTION

1. Field of the Invention

The present invention relates to a fragrance container, and more particularly to the improved fragrance container that features a simple structure and an easy and convenient operation and the fragrance container can achieve the effects of providing users a choice of fragrances and a diversified application at appropriate time conveniently.

2. Description of the Related Art

Fragrance is used extensively in different places including homes, offices or even in cars. Regardless of liquid or solid fragrances (such as perfume, essence oil, essence and ointment), the fragrances are contained and placed in a container for their convenient use.

Regardless of bottles, cans or boxes, the conventional containers used for containing fragrance generally come with the design of a single accommodation part, and the smell of the fragrance contained in the accommodation part can be dispersed to the outside from a preset opening of the container to achieve the aromatic effect in a space.

Although the aforementioned fragrance container with a single accommodation part can achieve the effects of containing the fragrance and dispersing the aromatic smell of the fragrance, yet such conventional fragrance container still has the following drawbacks:
1. The single accommodation part of the container just can contain one kind of fragrance, and the fragrance (such as perfume, essence oil, essence or ointment) contained in the container has a service life from a few weeks to a few months only, and thus the conventional fragrance container continues dispersing a single smell within the time period of the service life of the fragrance, not only giving a monotonous and dull sense of smell but also failing to achieve the expected effect of the fragrance.
2. To overcome the aforementioned drawback, users generally buy several fragrances of different smells contained in different containers, such that the containers can be opened or closed to disperse the fragrances from different containers alternately, so as to achieve the effect of changing the smell of the fragrance in a space. However, such arrangement not only increases the cost, but also requires a troublesome and inconvenient operation to change the fragrances.
3. Since each of the containers containing fragrance of a single smell, therefore the containers must be changed alternately. As time passes, the troublesome and inconvenient operation may result in a poor or low aromatic effect of the fragrances.

### SUMMARY OF THE INVENTION

Therefore, it is a primary objective of the present invention to overcome the aforementioned drawbacks by providing an improved fragrance container that can provide users a choice of fragrances and a diversified application at appropriate time conveniently.

To achieve the aforementioned objective, the present invention comprises a container body and a cover, wherein the container body has a plurality of chambers for containing fragrance; the cover is movably installed to the container body for opening and closing each chamber of the container body and has a guide passage communicated to the outside, and the guide passage is movable to a position corresponding to each chamber of the container body separately, so that the container body can contain fragrances of different smells in the chambers respectively, and the guide passage of the cover can be moved to a position corresponding to each chamber of the container body separately, and the smells of fragrances contained separately in each chamber of the container body can be dispersed to the outside immediately to achieve the effects of providing more choices of fragrances at appropriate time conveniently. The objectives, technical characteristics, measures, effects and advantages of the present invention will become apparent with the detailed description of preferred embodiment accompanied with the illustration of related drawings as follows.

### BRIEF DESCRIPTION OF THE DRAWINGS

FIG. 1 is a perspective view of a preferred embodiment of the present invention;

FIG. 2 is an exploded view of FIG. 1;

FIG. 3 is a cross-sectional view of one of the chambers of a container body corresponding to a guide passage in accordance with a preferred embodiment of the present invention; and

FIG. 4 is a cross-sectional view of each chamber of a container body being closed by a cover in accordance with a preferred embodiment of the present invention.

### DETAILED DESCRIPTION OF THE PREFERRED EMBODIMENTS

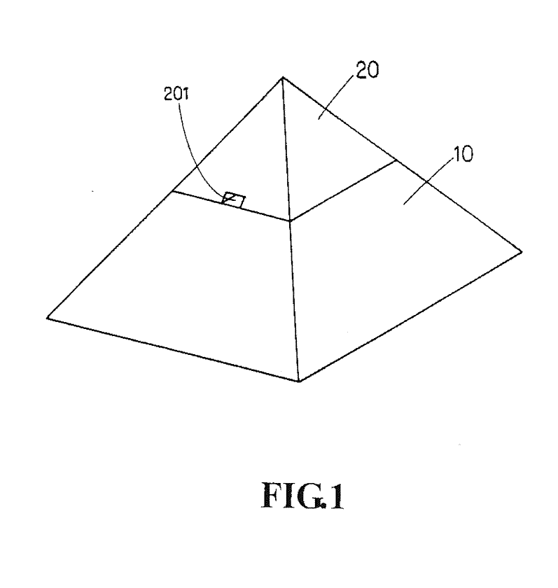

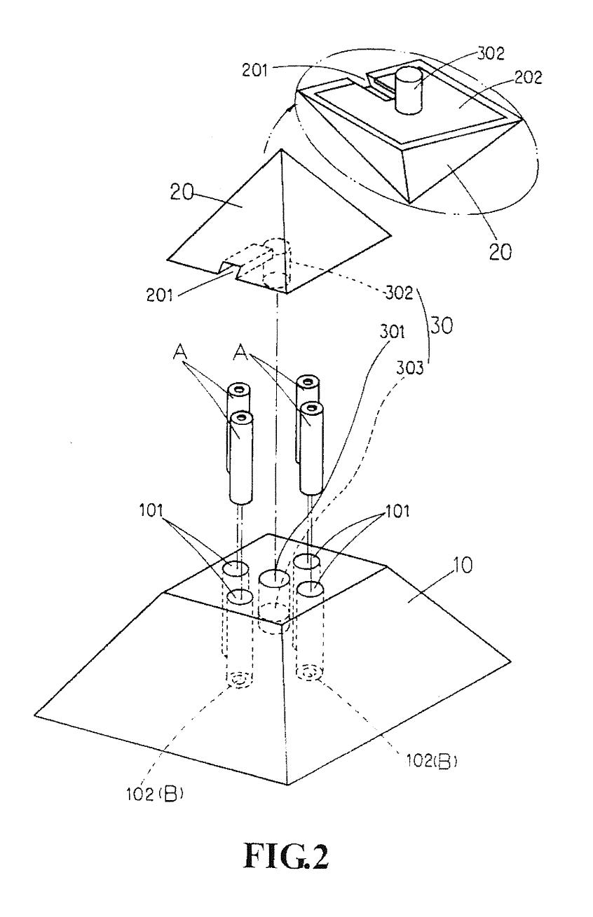

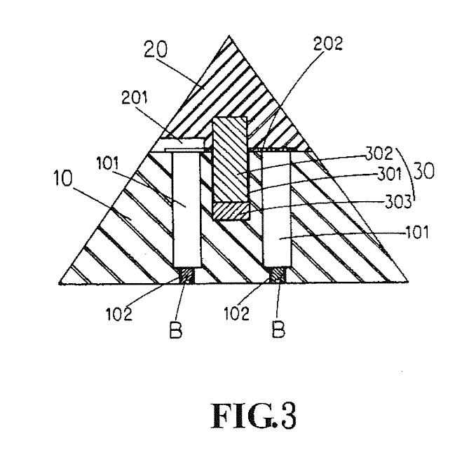

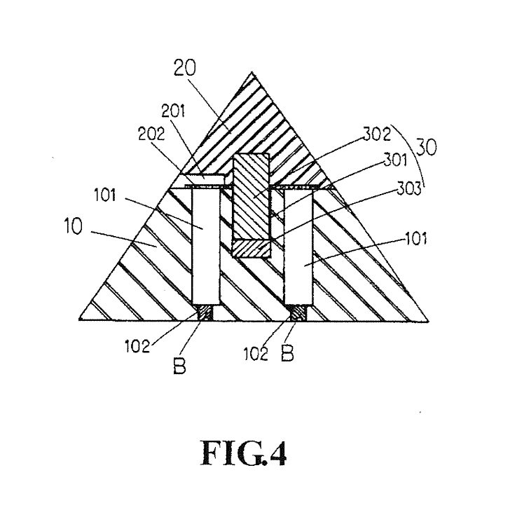

With reference to FIGS. 1 to 4 for an improved fragrance container of the present invention, the fragrance container comprises:
a container body 10, having a plurality of chambers 101, for containing liquid or solid fragrances;
a cover 20, movably installed to the container body 10, for opening and closing each chamber 101 of the container body 10, and the cover 20 having a guide passage 201 communicated with the outside and the guide passage 201 being movable to a position corresponding to each chamber 101 of the container body 10 separately, and having an airtight element 202 installed at the circumferential surface of the container body 10 and abutted against the container body 10 for sealing the chamber 101 of the container body 10;
a positioning device 30, installed between the container body 10 and the cover 20, and having positioning hole 301 formed on the container body 10 and a pivot shaft 302 pivotally installed to the positioning hole 301 formed on the cover 20, such that the pivot shaft 302 serves as a turning point, and the cover 20 can be turned freely with respect to the container body 10, and the guide passage 201 of the cover 20 can be aligned precisely with one of the chambers 101 of the container body 10 (as shown in FIG. 3), or the guide passage 201 may not be aligned with any chamber 101 in order to seal each chamber 101 completely by the airtight element 202 (as shown in FIG. 4).

Wherein, each chamber 101 of the container body 10 is provided for containing a solid or liquid fragrance directly or fragrance filled in the container A.

Wherein, each chamber 101 of the container body 10 has a guide hole 102 formed at the bottom of the chamber 101 and communicated to the outside, and a sealing element B is installed at the guide hole 102, such that after the sealing element B is removed, a cylindrical body (not shown in the figure) can be extended inwardly from the outside of the guide hole 102 to eject the fragrance container A outwardly from the chamber 101 for replacing the fragrance container.

Wherein, the positions of the positioning hole 301 and the pivot shaft 302 of the positioning device 30 can be exchanged, so that the positioning hole 301 is formed on the cover 20 and the pivot shaft 302 is disposed on the container body 10. In addition, the positioning hole 301 has a positioning element 303 installed therein, wherein the positioning element 303 and the pivot shaft 302 are metal components attracting one another, so that after the cover 20 is covered onto the container body 10, the pivot shaft 302 and the positioning element 303 are magnetically attracted to one another to enhance the positioning stability and strengthen the effect of the airtight element 202 for sealing the chamber 101.

In the aforementioned fragrance container of the present invention, each chamber 101 of the container body 10 contains fragrance, and after the cover 20 is covered onto the container body 10, the pivot shaft 302 acts as a turning point for free rotations, and thus the guide passage 201 is not aligned with any chamber 101 of the container body 10, and each chamber 101 is sealed completely by the airtight element 202 (as shown in FIG. 4). As a result, the smell of fragrance contained in each chamber 101 of the container body 10 will not be vaporized or dispersed to the outside easily. In addition, the cover 10 can be turned to align the guide passage 201 with one of the chambers 101 of the container body 10 precisely (as shown in FIG. 3). Since the remaining chambers 101 of the container body 10 are still sealed by the airtight element 202 of the cover 20, the smell of the fragrance contained in the chamber 101 aligned with the guide passage 201 can be dispersed to the outside successfully through the guide passage 201, so as to achieve the expected purpose and effect of using the fragrance.

In other words, each chamber 101 of the container body 10 can contain fragrance of a different smell, and the cover 20 can be turned freely to align the guide passage 201 precisely with any one of the chambers 101 of the container body 10, so that the smells of different fragrances contained in the chambers 101 can be vaporized and dispersed outwardly through the guide passage 201. Obviously, users can adjust the cover 10 freely as needed to achieve the effect of changing or selecting a different fragrance, and thus the invention can provide the users more choices of the fragrances and a diversified application at appropriate time conveniently.

Since the cover 20 and the container body 10 can be engaged or separated with each other simply, easily and conveniently, and the fragrance contained in each chamber 101 of the container body 10 can be supplemented or changed easily to facilitate the application without purchasing additional fragrance containers. Particularly, the invention can improve the utilization of the fragrance and provide users a better refreshed and diversified effect anywhere.

In summation, the present invention at least has the following advantages:
1. Simple structure with a low cost.
2. Easy operation for a convenient use.
3. Better choice of fragrances, diversified application, timeliness and convenience.
4. Enhanced effect of the fragrance for better refreshed and diversified effects anywhere.

What is claimed is:

1. A fragrance container, comprising:
a container body, having a plurality of chambers;
a cover, movably installed to the container body, for opening and closing each chamber of the container body.

2. The fragrance container of claim 1, wherein the cover has a guide passage communicated to the other, and the guide passage is movable to a position corresponding to each chamber of the container body separately.

3. The fragrance container of claim 1, further comprising an airtight element installed at a circumferential surface where the cover and the container body abut against each other and aligned precisely with each chamber of the container body.

4. The fragrance container of claim 1, wherein each chamber of the container body has a guide hole formed at the bottom of each chamber and communicated to the outside.

5. The fragrance container of claim 4, wherein each chamber of the container body has a sealing element installed at the guide hole at the bottom of the chamber.

6. The fragrance container of claim 1, further comprising a positioning device installed between the container body and the cover.

7. The fragrance container of claim 6, wherein the positioning device includes a positioning hole formed on the container body and a pivot shaft pivotally installed to the cover.

8. The fragrance container of claim 6, wherein the positioning device includes a positioning hole formed on the cover and a pivot shaft is pivotally installed to the positioning hole of the container body.

9. The fragrance container of claim 7, wherein the positioning hole contains a positioning element disposed therein, and the positioning element and the pivot shaft are metal components attracting one another.
